(19) United States
(12) Patent Application Publication
(10) Pub. No.: US 2002/0164367 A1
(43) Pub. Date: Nov. 7, 2002

(54) Botanical drug for increasing immunity and decreasing side effects of chemotherapy

(76) Inventor: Yaguang Liu, Flushing, NY (US)

Correspondence Address: Yaguang Liu, 67-08 168th Street, Flushing, NY, 11365, US

(21) Appl. No.: 09/798,047

(22) Filed: Mar. 5, 2001

(51) Int. Cl. A61K 35/78; A61K 9/127

(52) U.S. Cl. 424/450; 424/725

(57) ABSTRACT

This invention relates to new safe botanical drug, which is used for increasing immune function and decreasing side effects of chemotherapy.

Specifically, this invention provides a method for producing Oleanolic acid and BDI's polysaccharide.

### BACKGROUND OF THE INVENTION

1. Field of the Invention

This invention relates to a new pharmaceutical composition for increasing the immune function and decreasing side effects of chemotherapy. Chemotherapy includes anticancer and anti-viruses chemotherapy.

Specifically, this invention provides a new composition of a new botanical drug contained ligastrum lucidum and its active ingredients.

2. Description of Prior Art

A lot of anticancer medicines including chemical and antibiotics have effect to kill of cancer cells. But it also kills off some normal human cells. For example, Cyclopho-sphamide is a chemotherapeutic drug, which is highly effective against a wide range of human cancer.

Cyclophosphamide established a role in the treatment of some major cancer types including Lymphomas, Acute Lymphatic leukemia, Chronic Lymphatic Leukemia, Breast, Pulmonal, Ovarial cancer and Tumor or marrow mulciple, Osseous, Sarcoma etc. Unfortunately, Cyclophosphamide has toxicity, for example, it does damage to hemotopoietic organs, alimentary tract and decrease immune function. The toxicity of other anti-cancer medicines, for example, Fluorouracil, Mustine and 6-Mercaptopurine, etc. is higher than Cyclophosphamide.

Some antibiotics are effective anticancer drugs, for example, Adriamycin is used for treatment of some cancers including leukemia, gastric pancreatic, breast cancer, etc. A prime limit factor to the administration of Adriamycin is cardiotoxicity. The most serious side effect of Adriamycin administration is myocardial degeneration causing congestive heart failure. This acute cardiomyopathy may cause acute left ventricular dysfunction, arrhythmia and myocardial infarction. Adriamycin induced cardiomyopathy is thought to be permanent and rapidly progressive. Late cardipmyopathy develops in weeks, months or even years. Some patients were reported to have developed progressive cardiomyopathy two and two-half years after receiving this drug.

Obviously, to overcome toxicity of anti-cancer is very important. Many agents have been suggested to reduce or prevent toxicity of anticancer medicines. For example, Vitamin E and N-acetyl-L-Cysteine have also been reported to be effective in preventing Adriamycin cardiotoxicity. Although more recent research showed evidence to contradict these findings, in that neither of vitamin E and N-acetyl-L-Cysteine prevents adriamycin-induced cardiotoxicity.

The major antiviral drugs can inhibit viral replication but also inhibit some host cell function and possess serious toxicity. For example, Amantadine, Idoxuridine, Cytarabine, and Vidarabine are major antiviral drugs using in clinic now. Amantadine can inhibit myxovi-ruses, e.g, influenza A, rubella. The most marked toxic effects of Amantadine are insomnia, slurred speech, dizziness, ataxia and other central nervous system sign. Idoxuridine can inhibit the replication of herpes simplex virus in the cornea. However, DNA synthesis of host cells is also inhibited. Cytarabine can inhibit DNA viruses. But it also inhibits immune function in human. By weight it is about 10 times more effective than Idoxuridine, and it is also 10 times more toxic for host cell. Vidarabine can inhibit herpes-virus, but it is also produces more marked adverse gastrointestinal or neurologic side effects.

The immune system plays an important role in human body. The immune function normally protects human being from infections caused by viruses, bacteria, fungi, parasites and from developing cancer.

So far, no drugs have been succeeded to overcome toxicity of anticancer and anti-virus drugs and increasing immune function at the same time.

### DETAILED DESCRIPTION

According to the present invention, it has been found the bi-function of pharmaceutical composition of these ingredients have a significant effects in increasing the immune function and decreasing side effects of chemotherapy (BDI).

The results of research indicated that viral infection and developing cancer tends to cause disease only in individual whose immune function has been severely weakened. Individual with health immune function could control virus and cancer.

For reason given above, a new pharmaceutical composition (BDI) has a bi-function, which is decreasing side effects of chemotherapy and increasing immune function.

The following specific examples will provide detailed illustrations of methods of producing BDI according to the present invention and pharmaceutical dosage units containing BDI. Moreover, examples will be given of pharmaceutical testing performed with BDI which demonstrates its effectiveness in decreasing side effects of chemotherapy and increasing immune function. These examples are not intended, however, to limit or restrict the scope of the invention in any way, and should not be construed as providing conditions, parameters, reagents, or starting materials which must be utilized exclusively in order to practice the present invention.

The BDI contained two ingredients: Oleanolic acid and BDI's polysaccharides.

The structure of Oleanolic acid is shown as the following.

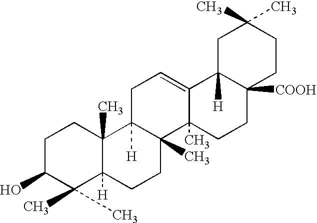

Molecular formula: C₃₀H₄₈O₃

Molecular weight: 456.71

Physical data:

a. mp: 310° C.

b. [α]²⁰+73.3° C.

c. white needles

d. UVλ^{EtOH} nm (ε):207 (4667)

IRλ^{KBr}μ:3.0 (OH), 5.9 (C═O).

### EXAMPLE 1

### Extract of Oleanolic Acid

The dried powder of Liqustrum lucidum Ait was extracted by 70% of ethanol at room temperature for 2 hours. The ethanol was recovered and still residue was obtained. The residue suspended in water. The water solution was extracted five times with petroleum ether. The petroleum extract was concentrated to syrup under vacuum and syrup was obtained. The syrup was passed through a column of silica gel and elute with petroleum. Elution was concentrated under vacuum and needles obtained. The needles were recrystallized and while needles were obtained.

### EXAMPLE 2

### Isolation of BDI's Polysaccharides

The dried powders of plant were extracted with hot water. The extract was filter. The ethanol was added to the extract. The precipitate was collected by centrifugation. The precipitate was dialyzed against running water. The precipitate was extracted with chloroform—isoamyl alcohol (4:1). The mixture was concentrated under vacuum. The still residue was chromatographed by a column of DEAE-sephadex A-25 and eluted with hot water. Eluate was concentrated under vacuum. The powders are BDI's polysaccharides.

### EXAMPLE 3

### Preparation of BDI-Containing Sterically Stabilized Liposomes (BDI-SSL)

Hydrogenated phosphatidylcholine (PC), phosphatidylglycerol (PGL), and phosphatidylserine (PS) were extracted from soybean. All above lipids were finally purified on silicic acid columns, shown to be pure by thin-layer chromatography and stored in chloroform in sealed ampoules under nitrogen until use. Phospholipids mixed with cholesterol (CHOL) and long-chain alcohol. The solvent was removed under reduced pressure by a rotary evaporator. Lipids were redissolved in the organic phase and reversed phase would be formed. BDI-containing phosphate-buffered saline (BDI was 3 mM in 0.1 M phosphate-buffered saline) was added at these lipid systems, and resulting two-phase system was sonicated 3 minutes until the mixture homogeneous that did not separate for at least two hours after sonicated. A typical preparation contined 3.3×10⁻³ M of phospholipid and 3.3×10⁻³ M of cholesterol in 1 liter of phosphate-buffered saline and 3 liters of solvent. BDI-SSL were sealed and sterilized. The size of the vesicles was determined. When PG/PC/CHOL were 1:4:5, diameter of liposomes was 20-50 nM (range). BDI-SSL was very stabilized in at least nine months. So far, many articles reported drug-containing liposomes. However, liposomes are not stabilized enough. Therefore, it is difficult to be used for pharmaceutical industry. In accordance with this invention, BDI-SSL is very stabilized in at least nine months. BDI-SSL, therefore, can be used in industry. BDI-SSL can enhance targeting and improve pharmaceutical activity of BDI. Also it is very important that all lipids are extracted from soybean. Therefore, it is very safe for human being. In fact, many articles reported lipids, which used for drug-containing liposomes, are syntheses by chemistry. However, synthetic lipids have some side effects, therefore, according to this invention, methods of preparing of BDI-SSL and phospholipids, which extracted from soybean, are very safe.

### EXAMPLE 4

### The Composition of BDI May Vary as Follows

TABLE 1

| | | Preferred composition |
|---|---|---|
| Ingredient | Weight percentage (%) | weight percentage (%) |
| Oleanolic acid | 30-70 | 50 |
| BDI's polysaccharides | 30-70 | 50 |

The dry ingredients of BDI, prepared in accordance with the present invention, may be incorporated in tablets, capsules and syrups by conventional methods which are not part of this invention. This invention will now be described with reference to its beneficial effects, as illustrated by the following tests:

### EXAMPLE 5

### The Influence of BDI on the Survival Rate of Myocardial Cells

Methods:

Hears were removed from 11 days embryos and were dissociated at 37 C. for 45 minutes with 0.25% trypsin (sigma, type III), 0.025% collagenase (sigma, type I), and 0.005% pancreatin (NBCO) prepared in calcium and magnesium free saline G containing 4% chicken serum. Then the tissue was dispersed into a single cell suspension in culture medium containing 5 g/ml DNAse I (sigma). Viable cell counts were determined by hemocyometer counting. Cells were dispersed into 60 mm culture disher (surface area 2000 mm²) at densities of 200 cells/mm².

Cultures were maintained in Ham's F-12K cln⁻ modified and supplemented with 5% fetal bovine serum. Tissue culture plates were incubated under at constant 5% CO₂ and 95% air at 37° C. Cells counted—all cells were counted in 20 randomly selected fields across the entire dish. A Zeiss microscope 25× objective having a field of view of 0.32 mm² was used for cell counting.

I. Reagents

1. CMF solution: 8.0 g NaCl, 0.4 g KCl, 0.15 g KH₂PO₄, 0.29Na₂HPO₄.7H₂O, 1000 ml distilled water, solution was sterilized by passage through a 0.22 millipore.

2. Phosphat-buffered saline (PBS): 10 ml 1M PO₄ buffer, 8 g NaCl, H₂O 1 liter, pH to 7.6.

3. PBS/BSA: PBS plus 1% bovine serum albumin.

4. Biotinylated Antibody (BA): ABC kit, 1 drop BA in 20 ml PBS/BSA.

5. Avidin Biotin Complex, ABC stain: ABC kit, 2 drops, Avidin DH, 2 drops Biotin-peroxidase, 10 ml PBS/BSA.

6. Diaminobenzidine reagents DAB: 5 mg diaminobenzidine tetrahydrochloride, 10 ml PBS, 0.004 ml 30% H₂O₂.

7. 0.3% H₂O₂ in methanol: 0.1 ml 30% H₂O₂ in 10 ml.

8. ABC kit purchased from Vector Laboratories, Inc., Burlingame, Calif.

9. Adriamycin purchased from Adria Laboratories, Inc., Columbus, Ohio.

II. Immunostaining Procedure

Monoclonal antibodies (diluted 1:1000 in PBS/BSA) were used. Immunostaining procedure used modified method of immunoperoxidase staining as follows.

1. Culture plates were added antibody of cardiac myosin before 3 hours for staining. Then culture plates continuous incubated 3 hours at 37° C.

2. Rinsed cell culture plates in CMF saline.

3. Fixed sections for 5 minutes in 50% methanol and 50% acetone.

4. Rinsed 3 times in methanol.

5. Incubated sections for 10 minutes in 0.3% H₂O₂ in methanol.

6. Incubated sections for 30 minutes in BA antibody solution.

7. Rinsed sections in PBS for 5 minutes with 2 times.

8. Incubated sections in ABC stain for 1 hour.

9. Rinsed sections in PBS for 5 minutes with 3 times.

10. Incubated sections in DAB-peroxide substrate solution for 5 minutes.

11. Rinsed sections for 10 minutes in water.

12. Counted stain for 30 seconds in 1% aqueous fast green, soaked in distilled water with 3 times.

13. Air dry cell cultures.

Cell determined by recording the color and the number of microscopic fields (250×). Normal cells were light green and dead cells were brown or very dark green.

Results:

The influence of BDI on Adriamycin induced damage of chick myocardial cell:

After chick myocardial cell was put into culture for 4 days, Adriamycin was added and remained in culture for 24 hours. Then ABC immunoperoxidase was used to determine survival rate. Data of Table 2 are shown that BDI significantly increases the survival rate of chick myocardial cell.

TABLE 2
The influence of BDI on the survival rate of myocardial cell

| | Survival rate | |
|---|---|---|
| Group | (living/taotalx 100%) | P |
| Adriamycin (10μ) | 36.0 ± 0.25 (*20) | — |
| Adriamycin (10μ) + BDI | 70.2 ± 0.60 (*20) | <0.01 |

*Indicated number of sampling
Concentration of BDI is 150 μg/ml.

### EXAMPLE 6

### Effects of BDI on Hemopoietic System

Effects of BDI on hemopoietic system were investigated. Results showed that BDI (ip) could markedly improve the recovery rate of hemopoieses in treatment mice by cyclophosphamide.

With increased cells in bone-marrow (BMC), endogenous colonies in spleen and higher ³H-TdR uptake in marrow and spleen. The level of serum colony stimulating factor (CSF) increased after injection. It was found that BDI protected the stem cells of bone marrow in mice from the killing effect of cyclophosphamide.

Pharmacological effects as illustrated by the following table: by means of the spleen colony assay technique the action of BD1 on bone marrow stem cells (CFU-S).

TABLE 3

| | Number of | | |
|---|---|---|---|
| Group | sample | Mean (CFU-S ± SD) | P |
| Control | 10 | 32.20 ± 3.0 | — |
| Cyclophosphamide | 10 | 7.6 ± 0.8 | <0.01* |
| BDI + Cyclophosphamide | 10 | 30.0 ± 0.3 | >0.50* |

*Different from control gronp

### EXAMPLE 7

### The Effect of BDI on Phagocytosis of Peritoneal Macrophage of Mice

Experimental Procedure:

Male mice weight 18-20 g were used in the experiments and were divided into treated (BDI) and control groups. The dosage of BDI was 5.5 mg/kg injected intraperitoneally. The control mice were injected with same volume of normal saline. These injections were repeated daily for 5 days, both treated and control group were injected intraperitoneally with cyclosphosphamide. The dosage of cyclophosphamide is 4.5 mg/kg.

The same experimental procedure for example 3, 4 and 5 in testing mice were used. Added 0.02 ml of 5% washed chick red blood cell suspension to 0.5 ml of the peritoneal exudates. Shook gently to mix and incubate at 37° C. for 5 minutes. Dipped two cover slips, closed to each other, in the above mixture and incubated for 30 minutes for the migration of the macrophages along the cover slips, fixed and stained with Sharma stain. Examined microscopically for:

Phagocytic rate—number of macrophages with phagocytized chick red blood cells per 100 macrophages counted.

Method of animal model is regular.

Results:

Pharmacological effects are as illustrated by the following table.

TABLE 4

| | Number of | Phagocytic | |
|---|---|---|---|
| Group | sample | (rate ± SD) | P |
| Control | 10 | 35.0 ± 0.40 | — |
| Cyclophosphamide | 10 | 8.5 ± 0.90 | <0.001 |
| BDI ± Cyclophosphamide | 10 | 31.0 ± 0.45 | >0.05 |

*Concentration of BDI and cyclophospharmide is the same as Example 2.

### EXAMPLE 8

### The Effect of BDI on White Blood Cells in Rats Treated by Cyclophosphamide

Action of BDI and cyclophosphamide on white blood cells was investigated by means of white blood cells assay. It was revealed that BDI protected white blood cells in rats from the killing effect of cyclophosphamide. Method of testing in animal is standard. The dosage of BDI and cyclophosphamide is the same as in Example 2. Time of treatment is 10 days. The results are listed below table:

TABLE 5

| | White blood cells x | Number of | |
|---|---|---|---|
| Group | 10³/cm³ ± SD | sample | P |
| Control | 15.0 ± 1.7 | 20 | — |
| Cyclophosphamide | 6.0 ± 0.70 | 20 | <0.001 |
| BDI + Cyclophosphamide | 13.0 ± 0.15 | 20 | >0.05 |

### EXAMPLE 9

### The Effect of BDI on Lymphoblastoid Transformation

By means of ³H-TdR liquid scintillation assay technique, the action of BDI on lymphoblastoid transformation was investigated method:

(1) Experimental procedure of animal is the same as in Example 2.

(2) Lemphoblastoid transformation test:

I. Reagents and Conditions for Cell Culture

a. Culture media—RPMI 1640, medium 199 minimal essential medium (Eagle).

b. Buffer—Hepes buffer, the final concentration at 37° C. was 25 mM, to maintain the pH of the medium at 7.31.

c. Serum—generally 15-205 fetal bovine serum was incorporated, for lymphocytes from mice, 5% was used.

d. Gaseous phase—5% CO₂ in air.

e. Cell concentration-generally 1-2 c 10⁶/ml.

f. Stimulants—20 μl/ml for phytohemagglutinin containing polysaccharide (PHA-M) or 10 μl/ml for polysaccharide-free purified phytohemagglutinin (PHA-P).

II. Measured by Liquid Scintillation

a. The conditions of cell culture are same as above. ³H-TdR was added after 48 hours of incubation at a final concentration of 1 μCi/ml and continued the incubation for 24 hours.

b. Washed the cells twice with cold normal saline and the erythrocytes were lysed. The intact lymphocytes were again washed once with cold saline. Spun down the lymphocytes and added 2 ml of 10% trichloroacetic acid to precipitate the protein.

Washed twice with normal saline. Added 2 ml of ethanol:ether (1:1) to wash once. 0.2 ml of formic acid was then added for digestion till the precipitate was dissolved.

c. Added 4 ml of scintillation fluid to 0.1 ml of the final sample and counted in a liquid scintillation counter.

Results are listed in the following table: (concentration of BDI and cyclophosphamide used is the same as that of Example 2).

TABLE 6

| | Number of | | |
|---|---|---|---|
| Group | sample | CPM ± SD | P |
| Normal | 10 | 1505 ± 130 | — |
| Cyclophosphamide | 10 | 600 ± 65 | <0.001 |
| Cyclophosphamide + BDI | 10 | 1250 ± 140 | <0.05 |

### EXAMPLE 10

### The Effect of BDI on Interleukin-2 (rIL-2)

The methods of determination rIL-2 were regular. The experimental data are listed in the following table.

TABLE 7

| | Number of | IL-2 | |
|---|---|---|---|
| Group | sample | (U/ml) ± SD | P |
| Normal | 10 | 85.0 ± 9.0 | — |
| Cyclophosphamide | 10 | 47.5 ± 5.5 | <0.01 |
| Cyclophosphamide + BDI | 10 | 72.0 ± 8.5 | <0.05 |

### EXAMPLE 11

### Effects of BDI on Immune Function of Human Blood Lymphocytes

20 old volunteers (60-70 years of age) and 10 healthy young persons participated in the experiment. 2 ml of venous blood, heparinized was obtained from each of the participants. The Study of the effects of BDI was carried out by using Eagle's Minimal Essential Medium MEM). MEM was supplemented with 0.125 ml of heat-inactivated fetal calf serum, 100 units of Penicillin and 0.1 mg of streptomycin per ml of medium. Culture medium was divided into treated (BDI) and control group. BDI was added to the culture medium of treatment group. The culture medium of control group was mixed with same volume as that of BDI of normal saline on the 72 hours of culture. The ³H-thymidine (³H-TdR) was added into all the cultures (2 μci/ml) for last 12 hours of culture. The cells were harvested on 0.45 μm filters, washed with phosphate buffer (ph 7.4) and bleached with H₂O₂. The filters were then dried and the incorporation of ³H-TdR into lymphocytes cell was measured by scintillation counter.

TABLE 8

| | Young (n = 20) | | Old (n = 20) | |
|---|---|---|---|---|
| Index | Control | BDI | Control | BDI |
| CPM | 8305 ± 900 | 8385 ± 216 | 7550 ± 809 | 1495 ± 156 |

| | | |
|---|---|---|
| T/C (%) | 101 | 198.1 |
| P | <0.01 | <0.01 |

According to Table 8, BDI is found to increase lymphoblastoid transformation of the old and young persons. However, BDI is found to add nothing to the young persons. In other words, BDI can increase human immune function in immunosuppressive state.

### EXAMPLE 12

### Effects of BDI on G-CSF and TNF-α

Blood cells of healthy volunteers (HV) and patients with leukemia (PL) cultured at same condition of Example 11. 1 ml of BDI (200μ/ml) added to each well of a 48-hole culture plate. Lipopolysaccharide (LPS, final concentration 10 μg/ml. Sigma, USA) used as a stimulant control. BDI or LPS was added to each well. The culture plate was incubated for 16 hours in the incubator which contained 5% CO₂ at 37° C. The supernatants were collected for analysis. G-CSF levels were measured using the ELISA system of Quantikin R & D (Buckinghamshire, UK, Cat No. DCS50); TNF-α levels in the collected supernatant were measured using the ELISA system of Endogen (Fleutus, Belgium, Code No. EH2-TNFA). Experiment was compared the differenced of G-CSF and TNF-α between healthy volunteers and patients with leukemia after treatment of BDI,

TABLE 9
Effects of BDI on G-CSF and TNF-α levels

| | G-CSF | | TNF-α | |
|---|---|---|---|---|
| Group | HV | PL | HV | PL |
| Before | 14.0 ± 2.0 | 24.8 ± 5.5 | 18.5 ± 2.5 | 24.3 ± 12.5 |
| After | 35.0 ± 6.5 | 103.0 ± 11.0 | 21.0 ± 2.8 | 180.5 ± 19.0 |
| T/C × 100% | 250.0 | 415.3 | 113.5 | 740.9 |

Data of Table 9 indicate that BDI can increase G-CSF and TNF-α of healthy volunteers (HV) and patients with leukemia (PL). More important is that increasing rate of PL group is more than HV group. BDI, therefore, is a very effective botanical drug used in increasing immune function for patients having cancer.

### EXAMPLE 13

### Toxicity of BDI

1. LD₅₀: 1200 mg/kg of injection in abdominal cavity in mice.

2. LD₅₀ of oral route was 5.1 g/kg.

3. Each dose for an adult was 20 mg. Used 50 kg as the average weight of one adult. The dosage was 0.3 mg/kg, it, therefore, was very safe.

The World Health Organization (WHO) established the classification of chemicals in 1973, according to relative toxicity. A chemical, which has a LD₅₀ of intragastric administration in mice >1 g/kg, is low toxic and >5 g/kg is basic no toxic. Therefore, BDI is a safe natural drug. This low toxicity was confirmed by sub-acute tests and absence of macroscopic lesions of the organ examined.

Results and experimental procedure of BDI to overcome toxicity of radiotherapy would be the same as in the examples above for chemotherapy.

The embodiment of the invention described here can be modified within the spirit and scope of the present invention. Numerous modifications and variations of the present invention are possible in light of the above teachings.

A pharmaceutical composition referred to as BDI, a process for producing BDI and furthermore its effect on decreasing side effects of chemotherapy and increasing the immune function as well as other effects studied by tests carried out by approved procedures have been described.

What is claimed as new and desired to be protected by Letters Patent is set forth in the appended claims:

1. A safe botanical drug (BDI) for increasing the immune function and decreasing side effects of chemotherapy comprises 30-70% by weight of Oleanolic acid and 30-70% by weight of BDI's polysaccharides

2. According to claim 1, a safe botanical drug BDI for increasing the immune function and decreasing side effects of chemotherapy is extracted from Ligustrum lucidum Ait or Ligustrum robustum BL.

3. A safe botanical drug BDI for increasing interleukemia 2 (rIL-2), tumor necrosis factor-α (TNF-α), colony stimulating factors (CSF), lymphoblastoid transformation, white blood cells, active of phagocytosis hemopoietic function and increasing the survival rate of myocardial cells comprises 30-70% by weight of Oleanolic acid and 30-70% by weight of BDI's polysaccharides.

4. A process for producing Oleanolic acid, which used for increasing immune function and decreasing side effects of chemotherapy, with claim 1 comprises:
a. Extracting said powder of Liqustrum lucideum Ait or Ligustrum robustum BL with 70% of ethanol at room temperature for 2 hours;
b. The ethanol was recovered and still residue was obtained;
c. The residue suspended in water;
d. The water solution was extracted five times with petroleum ether;
e. The petroleum extract was concentrated to syrup under vacuum and syrup was obtained. f The syrup was chromatographed by column of silica gel and elute with petroleum;
g. The elution was concentrated under vacuum and needles obtained;
h. The needles were recrystallized and white needles were obtained.

5. A process for producing BDI's polysaccharide, which used for increasing immune function and decreasing side effects of chemotherapy, with claim 1 comprises:
a. The dried powder of Ligustrum lucidum Ait or Ligustrum robustum BL was extracted with hot water;
b. The extract was filter;
c. The ethanol was added to the extract;
d. The precipitate was collected by centrigugation;
e. The precipitate was dialyzed against running water;
f. The precipitate was extracted with chloroform—isoamyl alcohol (4:1);
g. The mixture was concentrated under vacuum;
h. The still residue was chromatographed by a column of DEAE-sephadex A-25 and elute with hot water; and
i. Elute was concentrated under vacuum. The powder is BDI's polysaccharides.

6. A process of producing BDI-containing sterically stabilized liposomes (BDI-SSL) is comprising:
a. Phosphatidylcholine (PC), phosphatidylglycerol (PGL), and phosphatidylserine (PS) were purified from soybean;
b. PC, PGL, and PS were purified on silicic acid columns;
c. PC, PGL, and PS mixed with cholesterol (CHOL) and long-chain alcohol;
d. Lipids were dissolved in the organic phase and reversed phase would be formed;
e. BDI solution (BDI 3mM in 0.1 m phosphate-fubbered saline) was added at lipid systems and resulting two-phase system was sonicated 3 minutes; and
f. BD1-SSL was sealed and sterilized.
